(19) United States
(12) Patent Application Publication
(10) Pub. No.: US 2025/0114335 A1
(43) Pub. Date: Apr. 10, 2025

(54) METHODS AND COMPOSITIONS FOR THE TREATMENT OF EPILEPSY

(71) Applicant: ARIZONA BOARD OF REGENTS ON BEHALF OF THE UNIVERSITY OF ARIZONA, Tucson, AZ (US)

(72) Inventors: Michael F. Hammer, Tucson, AZ (US); Erfan Bahramnejad, Tucson, AZ (US)

(21) Appl. No.: 18/988,203

(22) Filed: Dec. 19, 2024

Related U.S. Application Data
(63) Continuation-in-part of application No. 18/952,827, filed on Nov. 19, 2024.
Application No. 18/952,827 is a continuation of application No. PCT/US2023/067230, filed on May 19, 2023.
(60) Provisional application No. 63/343,721, filed on May 19, 2022.

(51) Int. Cl. A61K 31/4245; A61K 31/4178; A61K 31/4184; A61P 25/08

(52) U.S. Cl. CPC A61K 31/4245; A61K 31/4178; A61K 31/4184; A61P 25/08

(57) ABSTRACT

Epilepsy is one of the most common neurological disorders. However, there are currently no drugs available to prevent and/or reduce the development of said disorder. Thus, compositions and methods described herein prevent and/or arrest the development of epilepsy. Specifically, angiotensin receptor blocker (ARB), such as candesartan, is described to treat epileptic patients and those at risk for developing epilepsy.

### CROSS-REFERENCES TO RELATED APPLICATIONS

This application is a continuation-in-part and claims benefit of U.S. patent application Ser. No. 18/952,827 filed Nov. 19, 2024, which is a continuation and claims benefit of PCT Application No. PCT/US2023/067230 filed May 19, 2023, which claims benefit of U.S. Provisional Application No. 63/343,721 filed May 19, 2022, the specifications of which are incorporated herein in its entirety by reference.

### FIELD OF THE INVENTION

The present invention features compositions and methods for the treatment of epileptic disorders.

### BACKGROUND OF THE INVENTION

Approximately 1 in 26 people will develop epilepsy at some point. Arizona has several higher-risk populations, including seniors, Native Americans, Latino communities, children, active military members, and veterans. Alarmingly, nearly half of the more than 77,000 Arizonans treated for active epilepsy continue to have seizures.

Currently, no drugs prevent or reverse the development of epilepsy, one of the most common neurological disorders. Given the high cost of bringing new medicines to market, drug repurposing offers the potential for significant savings in the time and cost of drug development. The present invention provides mechanistic insights into pathways that underlie epileptogenesis and determines which pathway effects are reversed by administering an angiotensin receptor blocker (ARB) with efficacy in treating epilepsy.

### BRIEF SUMMARY OF THE INVENTION

It is an objective of the present invention to provide compositions and methods that allow for mitigating the process of epileptogenesis, as specified in the independent claims. Embodiments of the invention are given in the dependent claims. Embodiments of the present invention can be freely combined with each other if they are not mutually exclusive.

Following the primary insult, whether mechanical or genetic, is a secondary injury cascade that includes a myriad of neuropathological processes, such as oxidative stress, neuroinflammation, astrocytosis, and disruption of the blood-brain barrier (BBB). The present invention demonstrates the therapeutic potential of angiotensin receptor blockers (ARB) in countering the secondary injury cascade.

In some embodiments, the present invention features a method of preventing or treating an epileptic condition (e.g., epilepsy) in a patient in need thereof. For example, the method may comprise administering a therapeutically effective amount of an angiotensin receptor blocker (ARB) to the patient. Non-limiting examples of epileptic conditions include but are not limited to pediatric epilepsy, a traumatic brain injury (TBI), Alzheimer's disease (AD), Parkinson's disease (PD), amyotrophic lateral sclerosis (ALS), Huntington's disease (HD), stroke, post-traumatic epilepsy, or temporal lobe epilepsy (TLE).

In other embodiments, the present invention may also feature a method of protecting the blood-brain barrier (BBB) in a subject in need thereof. The method comprises administering a therapeutically effective amount of an angiotensin receptor blocker (ARB) to the subject.

In some embodiments, the present invention may also feature a method of preventing a seizure in a subject in need thereof, the method comprising administering a therapeutically effective amount of an angiotensin receptor blocker (ARB) to the patient.

In other embodiments, the present invention features a composition comprising an angiotensin receptor blocker (ARB) for use in a method of treating an epileptic condition (e.g., epilepsy) in a patient in need thereof.

One of the unique and inventive technical features of the present invention is using an angiotensin receptor blocker (ARB; e.g., candesartan (CAN)) to prevent or slow the progression of epileptogenesis. Without wishing to limit the invention to any theory or mechanism, it is believed that the technical feature of the present invention advantageously provides for the treatment of epileptic conditions through the protection of the blood-brain barrier. None of the presently known prior references or work has the unique, inventive technical feature of the present invention.

Furthermore, the prior references teach away from the present invention. For example, angiotensin receptor blockers (ARBs) are currently indicated in hypertension, not epilepsy. Additionally, ARBs have no known epilepsy-related targets (e.g., ion channels or neurotransmitter receptors).

Furthermore, the inventive technical features of the present invention contributed to a surprising result. For example, in a mouse model of severe pediatric epilepsy, treatment of mice before seizures led to statistically significant delays in seizure onset, while treatment of mice at the time of seizure onset reduced seizure frequency and increased survival. These effects were robust in juvenile mice, as well as adult females and males.

Any feature or combination of features described herein is included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B show mouse lines and dosing regimens for B6-D/D juvenile and B6-D/+ adult mice. FIG. 1A shows survival curves for three mouse lines under study. Seizure onset for B6-D/+ heterozygotes is at ˜75 days, and survival declines to near 0% between 80 and 140 days; seizure onset for B6-D/D homozygotes is at ˜15-20 days, and survival declines to near 0% between 21 and 28 days and seizure onset for C3H-D/D mice begins ˜40-45 days, and survival declines to near 0% between 60 and 90 days. In FIG. 1B black bars indicate approximate seizure onset times for B6-D/D juveniles and B6-D adults. Treatment by injection or by oral dosing starts on day after first seizure for adults (shown in gray bars). There is a daily subcutaneous (s.c.) injection over 5 days for juveniles and a 10-day series for adults starting at seizure onset. Oral dosing for adults starts at seizure onset and continues for an extended period before collection, which corresponds to the age for VEH-treated mice to experience ˜20 TCs.

FIGS. 2A, 2B, 2C, and 2D show the lifetime survival, seizure frequency, and seizure gaps for adult mice. FIG. 2A shows boxplots of the lifetime survival for untreated and treated B6-D/+ females and males. FIG. 2B shows Kaplan-Meier survival curves for untreated and treated C3H-D/D male adults. Log rank test P-value 2.15×10<sup>−7</sup>, standardized effect size=0.79. FIG. 2C shows boxplots for post seizure onset seizure frequencies for untreated (VEH only) and treated B6-D/+ female and male adults. FIG. 2D shows boxplots of the number of seizure-free days between seizure bouts for untreated and treated B6-D/+ female and male adults (*P<0.05, ** P<0.01, *** P<0.001, **** P<0.0001; ns=not significant).

FIGS. 3A, 3B, 3C, and 3D show boxplots of the survival and seizure frequency of B6-D/+. FIG. 3A shows BD-6/+ survival, FIG. 3B shows C3H-D/D survival, FIG. 3C shows B6-D/+ post-onset seizure frequency, and FIG. 3D shows B6-D/D post-onset seizure frequency.

FIGS. 4A, 4B, 4C, and 4D show Kaplan-Meier survival curves for adults treated versus untreated mice. FIG. 4A shows B6-D/+ females, FIG. 4B shows B6-D/+ males, FIG. 4C shows C3H-D/D females, and FIG. 4D shows C3H-D/D males.

FIGS. 5A, 5B, 5C, and 5D shows boxplots for gap characteristics for B6-D/+ and C3H-D/D adults treated with CND. FIG. 5A shows B6-D/+ gap numbers, FIG. 5B shows B6-D/+ gap lengths in days, FIG. 5C shows C3H-D/D gap numbers, and FIG. 5D shows C3H-D/D gap length in days.

FIGS. 6A, 6B, 6C, and 6D show the lifetime survival and seizure frequency for juveniles treated with CND and PHT. FIG. 6A shows boxplots of the lifetime survival for juveniles treated with CND and PHT compared with untreated (VEH only) controls. FIG. 6B shows Kaplan-Meier survival curves for B6-D/D juveniles treated with CND compared with untreated (VEH only) controls. Log-rank test P-value 4.2×10⁻⁵, standardized effect size=0.79. FIG. 6C shows Kaplan-Meier survival curves for B6-D/D juveniles treated with PHT compared with untreated (VEH only) controls. Log rank test P-value 0.132 (*P<0.05, ** P<0.01, *** P<0.001, **** P<0.0001; ns=not significant). FIG. 6D shows boxplots of the post-onset seizure frequency for juveniles treated with CND and PHT compared with untreated (VEH only) controls.

FIG. 7 shows BBB paracellular permeability to sucrose is increased in untreated B6-D/+ female and male mice relative to those treated with CND after seizure activity. In situ brain perfusion with 14C-sucrose as a vascular permeability marker shows significantly elevated radioactivity represented by brain-to-perfusate radioactivity ratios (RBr %) in brains of untreated (VEH only) mice (light gray fill) after detection of seizures and compared with that of control (+/+) mice (white fill bar) and mice treated with CND (dark gray fill). Data are expressed as mean±SD of 5-6 animals per treatment group (*P<0.05, ** P<0.01, *** P<0.001, **** P<0.0001; ns=not significant).

FIGS. 8A, 8B, 8C, and 8D show the number of differentially expressed genes and pathways identified by RNAseq and pathway enrichment analysis for untreated and treated B6-D/D juvenile and B6-D/+ adult mice. FIG. 8A shows MDS plot of 500 most variable genes in untreated (lighter purple and red fill) and treated (darker purple and red fill) B6-D/D juveniles and B6-D/+ adult males, as well as age-matched controls (B6-+/+) (gray-scale fill). Individuals of each type are represented by gray-scale fill. Correspondence between numbers in circles and mouse phenotype and treatment status are shown in below the figure. FIG. 8B shows the number of DEGs (DEGs) identified in B6-D/D juveniles (top left) and B6-D/+ adult males (top right); (blue: down-regulated, salmon: up-regulated) and treated (mauve: down-regulated, green: up-regulated). The number of significantly enriched canonical pathways identified by IPA in B6-D/D juveniles (bottom left) and B6-D/+ adult males (bottom right); (blue: deactivated, salmon: activated) and treated (mauve: down-regulated, green: up-regulated). FIG. 8C shows bubble charts showing significantly enriched pathways (−log (P-value)>1.3) shared by B6-D/D juveniles and B6-D/+ adult males, color-coded by strength of activation (positive z-score≥+2.0) or deactivation (negative z-score≤−2.0). Asterisks indicate the most detrimental pathways (i.e., >6 detrimental effects). FIG. 8D shows a Venn diagram showing the number of canonical pathways shared or unique to untreated and treated B6-D/D juveniles and B6-D/+ adult males. Color scheme same as in (FIG. 8B).

FIG. 9 shows ARB-dosing regimes for juvenile and adult mice. Gray bars indicate the approximate start and end dosing times based on variable seizure onset and survival time, respectively. Black bars indicate hard start dosing times and/or certain periods of dosing based on a range of typical survival times.

FIG. 10 shows B6 Adult Male Survival and Seizure Frequency in untreated versus ARB-treated Late or ARB-treated Early Groups.

FIG. 11 shows a B6 Adult Male Gap (e.g., between seizures) Analysis in Untreated versus ARB-treated Late or ARB-Treated Early Groups.

FIG. 12 shows male lifetime seizures in untreated versus ARB-treated (i.e., a treated Late Group).

FIG. 13 shows B6 Adult Male Age at Onset and Mode of Death in Untreated versus ARB-Treated Late or ARB-Treated Early Groups; CSE=convulsive status epilepticus, SUD=SUDeP, DEC=decompensated.

FIG. 14 shows B6 Adult Male Survival in untreated versus ARB-Treated.

FIG. 15 shows B6 Juveniles in untreated versus ARB-or Phenytoin (PHT)-Treated Group.

FIG. 16 shows B6 Juvenile Survival in untreated versus ARB-or Phenytoin Treated Groups.

FIG. 17 shows C3H Male and Female Adults Survival in Untreated versus ARB-Treated Late Groups.

FIG. 18 shows that the results described herein are robust to strain, sex, and life stage.

FIG. 19 shows the extent of blood-brain-barrier disruption (BBBD) in untreated heterozygous (D/+) pre-seizure mice is elevated in both sexes relative to wildtype controls, although to a lesser extent in females. Treated pre-seizure mice show reduced BBBD, with permeability returning to baseline. Post-seizure mice of both sexes show a large increase in BBBD; however, permeability returns to near baseline in treated mice of both sexes.

FIGS. 20A and 20B show the effect of seizures on gene expression across the genome (transcriptome) in untreated homozygous D/D juvenile mice and changes in gene expression in response to treatment. FIG. 20A shows treated juveniles have fewer dysregulated genes than wildtype controls. FIG. 20B shows a pathway analysis indicates that untreated post-seizure juveniles activate a number of pathways involved in neuroinflammation, astrocytosis (fibrosis), cellular remodeling, and mitochondrial dysfunction. These pathways are returned to physiological baseline in treated juveniles, while mitochondrial function is enhanced.

Referring to the figures, results of statistical summaries are expressed as mean±SD. Kaplan-Meier survival curves were used to test for differences in survival. Unpaired t-tests were used to test for differences in survival, and chi-square tests were applied to test for differences in modes of deaths. In cases where groups did not have the same variance, two-sample t-tests were performed.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. The term does not denote a particular age or sex. Thus, adult, children, and newborn subjects, as well as fetuses, whether male or female, are intended to be included. In one embodiment, the subject is a mammal (e.g., a human) having a disease, disorder, or condition described herein. In another embodiment, the subject is a mammal (e.g., a human) at risk of developing a disease, disorder, or condition described herein. A “patient” is a subject afflicted with a disease or disorder. In certain instances, the term patient refers to a human.

The terms “treating” or “treatment” refers to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder, or of one or more symptoms thereof. In certain cases, the beneficial effects that a subject derives from a prophylactic or therapeutic agent do not result in a cure of the disease or disorder.

The term “effective amount” as used herein refers to the amount of a therapy (e.g., an angiotensin receptor blocker (ARB)) that is sufficient to reduce and/or ameliorate the severity and/or duration of a given disease, disorder, or condition and/or a symptom related thereto. This term also encompasses an amount necessary for the reduction or amelioration of the advancement or progression of a given disease (e.g., epileptic condition), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of an angiotensin receptor blocker (ARB) herein is an amount sufficient to provide a therapeutic benefit in the treatment or management of an epileptic condition or to delay or minimize one or more symptoms associated with the epileptic conditions. A therapeutically effective amount of an angiotensin receptor blocker (ARB) described herein means an amount of therapeutic agent, alone or in combination with other therapies, which provides a therapeutic benefit in the treatment or management of epileptic conditions. The term “therapeutically effective amount” can encompass an amount that improves overall therapy, reduces or avoids symptoms or causes, or enhances the therapeutic efficacy of another therapeutic agent.

The terms “administering,” and “administration” refer to methods of providing a pharmaceutical preparation to a subject. Such methods are well known to those skilled in the art and include, but are not limited to, administering the compositions intranasally, parenterally (e.g., intravenously and subcutaneously), by intramuscular injection, by intraperitoneal injection, intrathecally, transdermally, extracorporeally, topically or the like.

As used herein, “epileptogenesis” refers to the process between an initial injury (latent phase), the development of an epileptic condition (acute phase), and the progression of epilepsy after it is established (chronic phase).

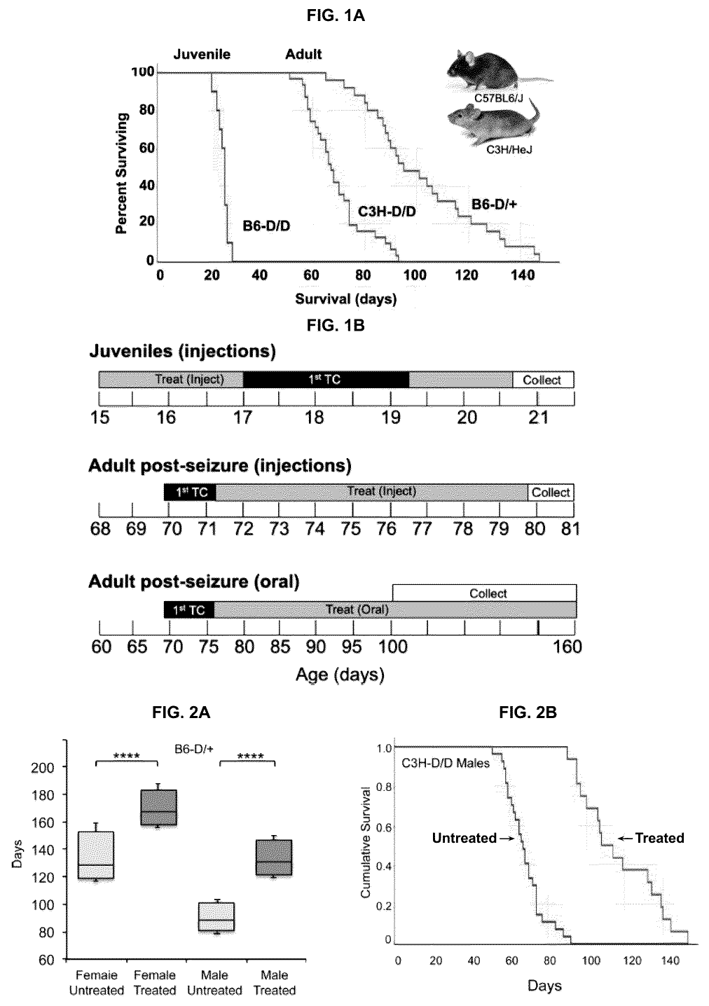

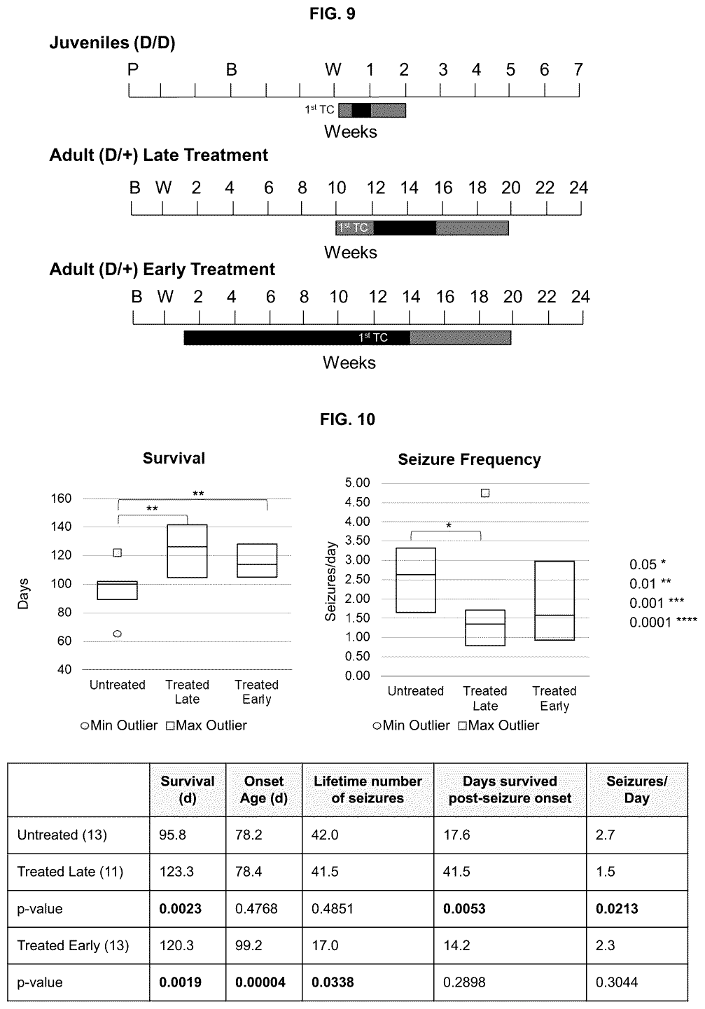

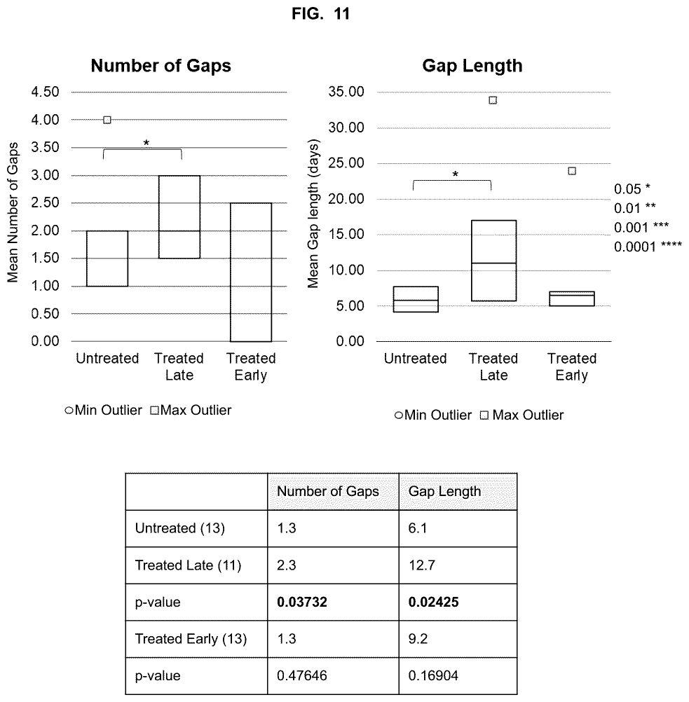

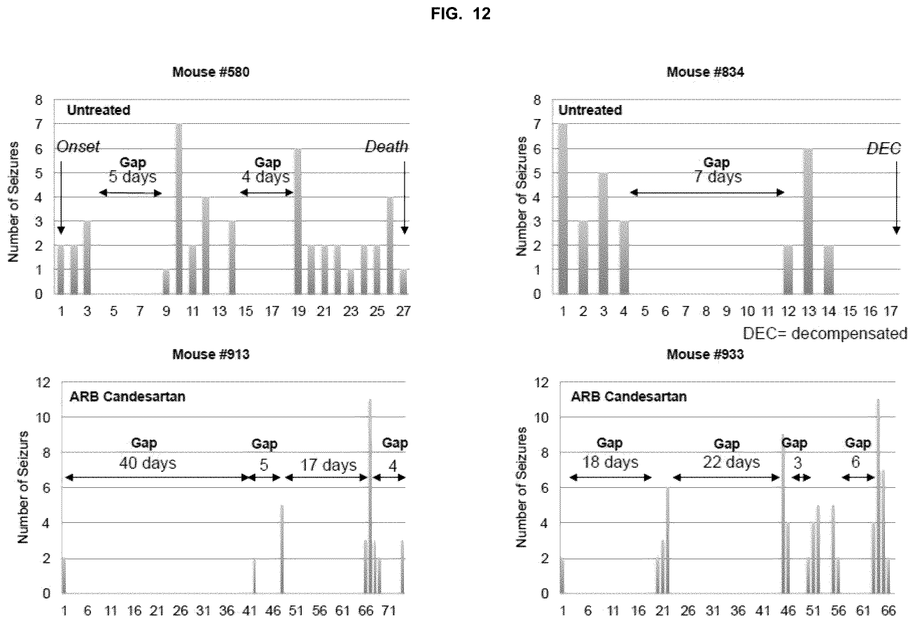

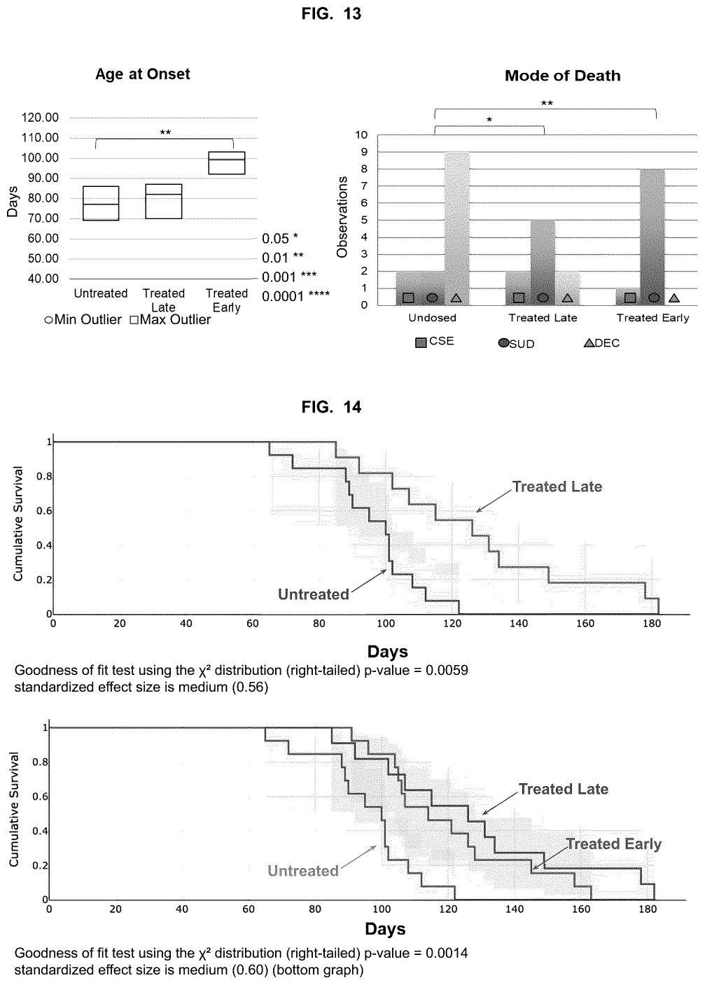

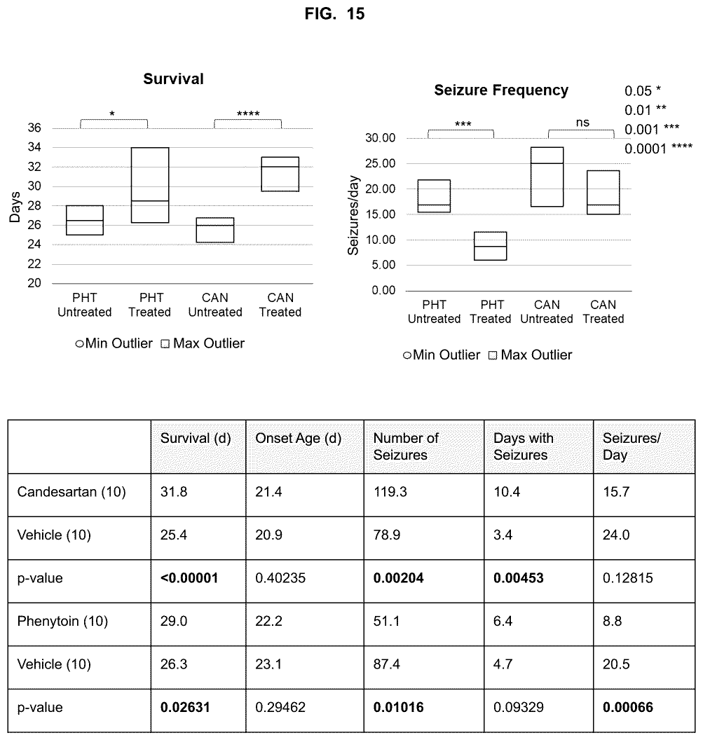

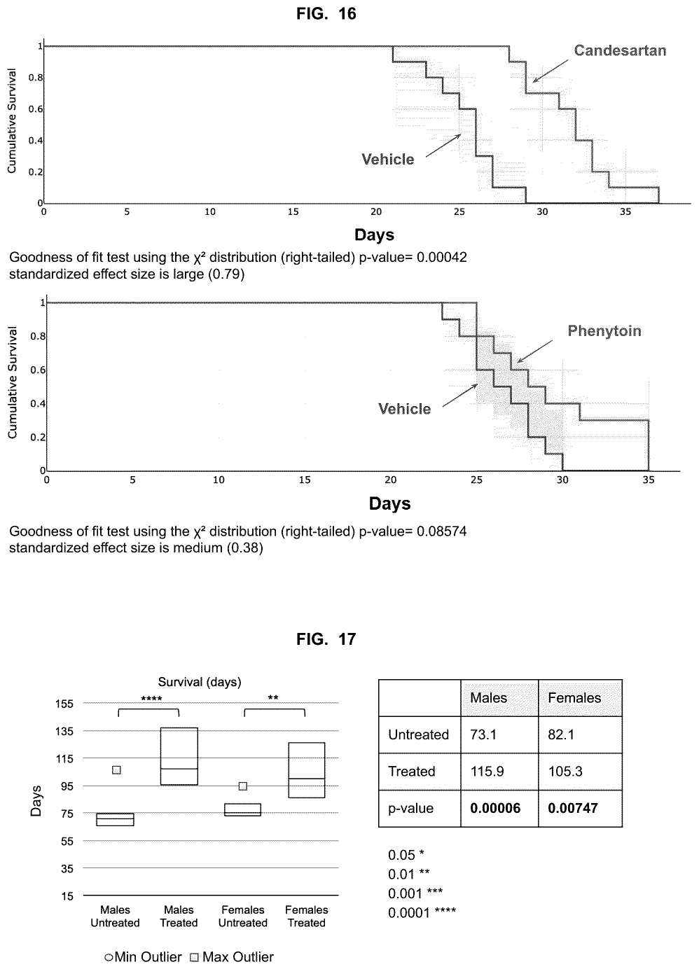

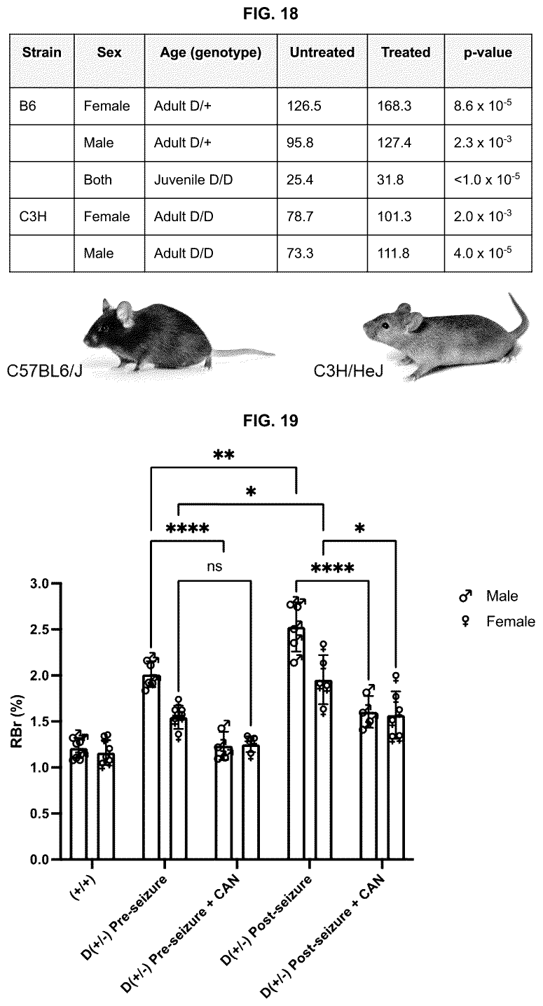

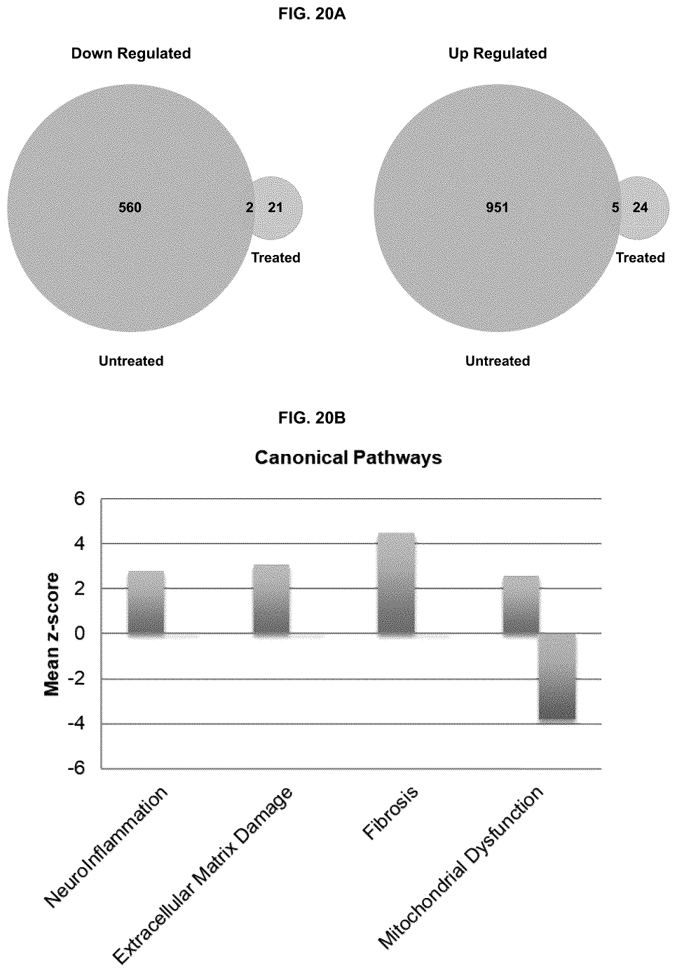

Referring now to FIGS. 1A-20B, the present invention features compositions and methods for the treatment of epileptic disorders (e.g., epilepsy).

In some embodiments, the present invention features a method of preventing or treating an epileptic condition in a patient in need thereof. The method may comprise administering a therapeutically effective amount of an angiotensin receptor blocker (ARB) to the patient.

In some embodiments, the present invention may also feature a method of preventing or treating epilepsy in a patient in need thereof. The method may comprise administering a therapeutically effective amount of an angiotensin receptor blocker (ARB) to the patient.

In some embodiments, the present invention may also feature a method of protecting the blood-brain barrier (BBB) in a subject in need thereof. The method comprises administering a therapeutically effective amount of an angiotensin receptor blocker (ARB) to the subject. In some embodiments, the present invention features a method of maintaining blood-brain barrier function in a subject in need thereof; the method may comprise administering a therapeutically effective amount of an angiotensin receptor blocker (ARB) to the subject.

Without wishing to limit the present invention to any theories or mechanisms, it is believed that compositions (e.g., sartans; e.g., candesartan) that inhibit angiotensin II receptor type 1 (ATIR) and/or activate peroxisome proliferator-activated receptor-gamma (PPARγ) are efficacious in modifying and/or arresting the development of epilepsy (i.e., an epileptic disorder). Specifically, angiotensin receptor blockers (ARB; e.g., candesartan (CAN)) prevent or slow the progression of epileptogenesis through a multi-target mechanism involving genes affecting inflammatory-immune response pathways and those maintaining the integrity of the BBB.

In some embodiments, the present invention may also feature a method of preventing a seizure in a subject in need thereof, the method comprising administering a therapeutically effective amount of an angiotensin receptor blocker (ARB) to the subject. In some embodiments, the present invention may also feature a method of treating a disease that causes a seizure in a subject in need thereof by administering a therapeutically effective amount of an angiotensin receptor blocker (ARB) to the subject.

In some embodiments, the angiotensin receptor blocker (ARB) comprises a sartan or a derivative thereof. Non-limiting examples of sartans may include, but are not limited to candesartan, losartan, valsartan, irbesartan, telmisartan, eprosartan, azilsartan, olmesartan, or derivatives thereof. In some embodiments, the angiotensin receptor blocker (ARB) is candesartan. In other embodiments, the ARB may be azilsartan, eprosartan, irbesartan, losartan, olmesartan, telmisartan, or valsartan. Other angiotensin receptor blockers (ARBs; e.g., other sartans or derivatives thereof) may be used in accordance with the present invention.

As used herein, an “epileptic condition” may refer to a condition of the brain characterized by repeated seizures. A seizure is usually defined as a sudden alteration of behavior due to a temporary change in the electrical functioning of the brain. As used herein, “epilepsy” refers to a condition of recurrent, unprovoked seizures, but may include abnormal brain activity associated with higher risk of unprovoked seizures.

In some embodiments, the epileptic condition is pediatric epilepsy. In other embodiments, the epileptic condition is traumatic brain injury (TBI) or other neurodegenerative diseases (e.g., Huntington's Disease, Alzheimer's Disease, or Parkinson's Disease). In some embodiments, the epileptic condition may derive from a stroke. Other diseases that cause seizures, e.g., with age, may be prevented or treated with methods described herein.

The present invention is not limited to the aforementioned diseases and may also encompass a range of brain disorders, e.g., neurodegenerative diseases, including Alzheimer's disease (AD), Parkinson's disease (PD), amyotrophic lateral sclerosis (ALS), Huntington's disease (HD), stroke, post-traumatic epilepsy, and temporal lobe epilepsy (TLE). These disorders could potentially benefit from treatment with angiotensin receptor blockers (ARBs), such as candesartan (CND). For example, in some embodiments, the present invention features a method of preventing or treating a neurodegenerative diseases, e.g., Alzheimer's disease (AD), Parkinson's disease (PD), amyotrophic lateral sclerosis (ALS), Huntington's disease (HD), stroke, post-traumatic epilepsy, and temporal lobe epilepsy (TLE), in a patient in need thereof. The method may comprise administering a therapeutically effective amount of an angiotensin receptor blocker (ARB) to the patient.

The methods and/or treatments described herein may reduce seizure frequency in a patient. Additionally, the methods and/or treatments described herein may improve memory, learning, and alertness. Without wishing to limit the present invention to any theory or mechanism, it is believed that the use of the treatments herein improves the quality of life of the patient.

In some embodiments, the patient is a child. In some embodiments, the patient is an adult. Without wishing to limit the present invention to any theory or mechanism, treatments described herein may be used in a patient as young as one-years-old and may be beneficial in treating or preventing early child epilepsies.

In some embodiments, the ARB is delivered orally, e.g., in a pill or liquid form. In some embodiments, the ARB is delivered daily.

The present invention features a composition comprising an angiotensin receptor blocker (ARB) for use in a method of treating an epileptic condition in a patient in need thereof. In some embodiments, the present invention features a composition comprising an angiotensin receptor blocker (ARB) for use in a method of treating epilepsy in a patient in need thereof. In other embodiments, the present invention may also feature a composition comprising an angiotensin receptor blocker (ARB) for use in a method of treating a disease that causes a seizure in a patient in need thereof.

The present invention may also feature a composition comprising an angiotensin receptor blocker (ARB) for use in a method of preventing an epileptic condition in a patient in need thereof. In some embodiments, the present invention features a composition comprising an angiotensin receptor blocker (ARB) for use in a method of preventing epilepsy in a patient in need thereof. In other embodiments, the present invention may also feature a composition comprising an angiotensin receptor blocker (ARB) for use in a method of preventing a seizure in a patient in need thereof.

In some embodiments, the present invention features a composition comprising an angiotensin receptor blocker (ARB) for use in a method of protecting the blood-brain barrier (BBB) in a patient in need thereof. In some embodiments, the present invention features a composition comprising a sartan for use in a method of protecting the blood-brain barrier (BBB) in a patient in need thereof.

In some embodiments, the present invention may further feature a composition comprising a sartan for use in a method of preventing or treating an epileptic condition (e.g., epilepsy) in a patient in need thereof. In some embodiments, the present invention features a composition comprising a sartan for use in a method of preventing a seizure in a patient in need thereof. In other embodiments, the present invention features a composition comprising a sartan for use in a method of treating a disease that causes a seizure in a patient in need thereof.

In some embodiments, the presenting invention features the use of a compound comprising an angiotensin receptor blocker (ARB; e.g., a sartan, e.g., candesartan) in the manufacture of a medicament for the treatment of an epileptic condition (e.g., epilepsy).

### Example 1

The following is a non-limiting example of the present invention. It is to be understood that said example is not intended to limit the present invention in any way. Equivalents or substitutes are within the scope of the present invention.

A healthy brain requires a healthy blood-brain barrier. The BBB controls the blood-to-brain exchange of nutrients, xenobiotics, blood components, and cells, ultimately maintaining the optimal brain milieu necessary for physiologic neuronal function. Disturbance of the blood-to-brain equilibrium can be a cause or consequence of central nervous system diseases, like epilepsy. Targeting of the damaged or dysfunctional BBB may represent a therapeutic approach to reduce seizure burden.

A mouse model with a knockin gain-of-function mutation (Scn8a-N1768D) in the voltage-gated sodium channel, NaV1.6 (encoded by SCN8A) leads to excess excitability in the brain and the production of seizures in virtually 100% of mice after an initial latent phase. Utilizing RNAseq of hippocampal tissue, different cellular and signaling pathways are altered in the latent phase, at the time of seizure onset, and during the chronic phase. Pathway analysis tools predicted that ARBs could potentially offset the effects of several of these pathological processes, including activation of peroxisome proliferator-activated receptor (PPAR) signaling and deactivation of Bone Morphogenetic Protein (BMP) and Transforming Growth Factor-β (TGF-B) signaling-pathways involved in regulating the BBB.

To determine whether blockade of the AT1 receptor with candesartan increased survival, mice were observed from an early age until the first tonic-clonic seizure (TC). Specifically, the aim was to determine whether candesartan increases the number of days mice lived after an initial tonic-clonic seizure (TC). Throughout the observation, the mice were orally given 4 mg/kg/day of candesartan via a peanut butter pellet.

Mice treated with candesartan (CAN) at different stages of epileptogenesis demonstrated statistically significant: 1) delay of age at seizure onset, 2) increased adult survival and a reduction in seizure frequency, and 3) increased juvenile survival.

Additionally, the effects of candesartan on the blood-brain barrier (BBB) and gene expression patterns both before and after seizure onset have been determined. Candesartan acts to prevent seizure onset and improve outcomes post-seizure, and it does so by protecting the BBB. Without wishing to limit the present invention to any theory or mechanism, it is believed that candesartan acts by a combined mechanism of action-both as an ATIR antagonist and a PPARγ (peroxisome proliferator-activated receptor-gamma) activator. These pathways are known to be involved in maintaining BBB function.

### Example 2

The following is a non-limiting example of the present invention. It is to be understood that said example is not intended to limit the present invention in any way. Equivalents or substitutes are within the scope of the present invention.

Example 2 demonstrates the efficacy of candesartan (CND), an FDA-approved ARB for hypertension, in preventing BBB dysfunction and mitigating epileptogenesis in the Scn8a mouse model, which includes the N1768D variant in heterozygous (D/+) and homozygous (D/D) forms on C57BL/6J and C3H/HeJ strain backgrounds.

Mouse strains: The C57BL/6J-N1768D congenic mouse (B6-D/+) was used for phenotyping, RNAseq, and BBB studies. In addition, a hybrid strain that was produced by backcross breeding to transfer the Scn8a-N1768D allele from the B6 to the C3H/HeJ strain background was used to further assess the effect of CND on survival and seizure frequency. After constructing an F1 by crossing male B6-D/+ heterozygotes to female C3H/HeJ, five rounds of backcrossing (N6) produced a 2.5% B6 and 98.5% C3H/HeJ-N1768D line (C3H-D/+). Sister-brother mating maintained the 98.5% C3H background. This line (referred to here as C3H) was observed to have an altered phenotype in terms of survival of the homozygote (C3H-D/D), which typically only survive for ˜25 days after a series of ˜60-80 tonic-clonic seizures (TCs) beginning at ˜15-20 days of age (FIG. 1A). C3H-D/D mice have a delayed seizure onset (˜40-45 days) and an extended survival (65-70 days) with intermittent bouts of TCs (FIG. 1A).

Phenotyping: Female and male mice on both the B6 and C3H backgrounds were housed in sex-specific groups of 3-4 per cage in a pathogen-free mouse facility with a 14 h light/10 h dark cycle (lights turned on at 5 am). A 24/7 video monitoring system was utilized to collect seizure data, with infrared illumination to monitor behavior during the dark period. Seizures were counted as individual TCs. Adult mice were followed from the age of 6 weeks, a period of time well before seizure onset, in order to identify the day of seizure onset. A seizure bout was defined as a cluster of seizures on consecutive days, and a gap as a seizure-free period of 3 days or more.

### Drug Administration:

Preparation of CND suspension for injections and oral dosing: Juvenile B6-D/D and adult B6-D/+ mice were given subcutaneous (s.c.) injections with either CND (2-4 mg/kg/day; doses that fall within the FDA-approved range for CND in both adults and children) or vehicle (VEH) (Sigma-Aldrich) (FIG. 1B). Oral dosing of adults was performed by thoroughly mixing 160 μl of suspension containing 1.28 mg of CND or VEH with 5 g of pure (organic) peanut butter in a petri dish. Mice were given 1 g pellet per mouse each day. Pellets were weighed each day to record the amount consumed, and 24/7 video was used to verify that each mouse was consuming the pellet. An average of 0.79 g (±0.36 g) was consumed by an adult mouse (˜20-25 g), corresponding to 9.1±4.2 mg/kg/day. No correlations were found for the amount of CND consumed and lifetime survival or number of TCs.

Survival and seizure frequency studies: B6-D/D mice were injected with CND (n=10) or VEH (n=10) at 15 days of age (P15) and followed to monitor TCs by 24/7 video recording until death (FIG. 1B). A separate set of 10 B6-D/D mice was administered a daily s.c. injection of 25 mg/kg/day phenytoin (PHT) suspension or VEH (US Pharmacopeia, Sigma-Aldrich). Oral administration of CND or VEH was initiated at the time of seizure onset (range P75-P85) for adult B6-D/+ females (N=10) and males (N=10) (FIG. 1B). Mice were monitored for seizures by 24/7 video recording until the time of death.

Transcriptome studies: B6-D/D and wild-type (+/+) (n=3) were administered CND or VEH by s.c. injection for a period of 5 days starting at P15 and sacrificed for removal of hippocampal tissue (n=12) (FIG. 1B). Adult B6-D/+ male (n=3) mice were administered CND or VEH pellets as described above at the time of seizure onset and monitored for seizures for an extended period before sacrifice and removal of hippocampal tissue (100-160 d) (i.e., to match the age at which a VEH-treated male B6-D/+ experience ˜20 TCs) (FIG. 1B).

Blood-brain barrier studies: B6-D/+ females (n=6) and males (n=6) were administered CND (2-4 mg/kg/day) or VEH by s.c. injection at the time of seizure onset for a period of 10 days (FIG. 1B). On the 11th day, these two groups of mice were subjected to BBB analysis as described in the next section.

Blood-brain barrier analysis: Changes in BBB integrity were assessed by enhanced brain accumulation of 14C-sucrose (PerkinElmer Life and Analytical Sciences, Boston, MA). In situ perfusion with radiolabeled sucrose was performed. Briefly, mice were anesthetized with ketamine/xylazine and heparinized to ensure anticoagulation. An incision was made in the neck, and the right carotid artery was exposed and cannulated. Following cannula placement, the mouse was perfused with an artificial plasma solution warmed to 37° C. and continuously oxygenated containing [¹⁴C] sucrose (Specific Activity=0.5 mCi/ml) delivered via a slow-drive syringe pump. After 10 min of perfusion, the cannulae were removed, and the animal was decapitated. The brain was rapidly removed, and cerebral hemispheres were sectioned. Radioactivity of [14C] sucrose was measured by liquid scintillation counting. Results were reported as picomoles of radiolabeled sucrose per milligram of brain tissue (RBr; pmol/mg tissue), which is equal to the total amount of ¹⁴C-sucrose in the brain (R_{Brain}; dpm/mg tissue) divided by the amount of radioisotope in the perfusate (R_{Perfusate}; dpm/pmol) (equation 1):

$$RBr = \frac{{R}_{Brain}}{{R}_{Perfusate}} \qquad ( Equation 1 )$$

RNA sequencing: Hippocampal tissues from 21 mice were obtained according to a sampling strategy that compared (1) untreated (VEH only) and treated B6-D/D (n=3) with age-matched B6-+/+ controls (n=3 per group), (2) untreated (VEH only) male B6-D/+ mice that had experienced ˜20 TCs (n=3 per group) with age-matched B6-+/+ controls (n=3 per group), and (3) male B6-D/+ mice that were treated with CND for an extended period (n=3 per group). Brains of treated and untreated mice were dissected to yield tissue samples from the hippocampus. Bulk tissue was stored in RNALater (Qiagen, Valencia, CA) at −80 degrees centigrade. The technique for analyzing hippocampal gene expression was performed. Briefly, RNA was isolated from hippocampal tissue, and initial QC was performed. Libraries were constructed using a stranded mRNA-Seq Kit, and average fragment size was assessed. After concentrations were determined with an adaptor-specific qPCR kit, equimolar samples were pooled and clustered for sequencing on a Novaseq instrument (Illumina). Sample data were demultiplexed, trimmed, and quality filtered, and Fastq files were splice-aligned against the GRCh37 reference genome using STAR aligner version 2.5.2b. Gene expression counts were obtained using htseq-count version 0.6.1. Both splice alignment and counting were performed with Ensembl Annotation of the NCBI reference genome, and raw counts were analyzed with edgeR version 3.16.5.

Data and pathway analysis: Results of statistical summaries were generally expressed as mean±SD. Kaplan-Meier survival curves were used to test for differences in survival. In cases where groups did not have the same variance, two-sample t-tests were performed. A ‘perturbation signature’ approach was used to identify genome-wide differences in transcript abundance between transgenic mice (i.e., D/+, D/D, or +/+) that were untreated (VEH) or treated with CND. Differential expression analysis was performed. Briefly, the exactTest function was utilized in edgeR, and gene expression counts were first normalized using the calcNormFactors function. DEG analysis was performed on treatment versus control groups at two different ages using three biological replicates per group, which has been shown in power analyses to be sufficient to yield a true positive rate greater than 80% under the conditions used here. Multidimensional scaling (MDS) plots were constructed using the ‘plotMDS’ function in edgeR, which plots samples on a 2D scatterplot so that distances on the plot approximate the typical log 2 fold changes between samples. All significant differentially expressed genes (DEGs) (false discovery rate [FDR]<0.05) were analyzed with Ingenuity® Pathway Analysis (IPA) to identify biological pathways that were significantly activated or deactivated as compared with controls and to identify putative upstream transcriptional regulators (Qiagen, Hilden, Germany). Rather than focusing on any single gene, bioinformatic analyses of our RNAseq data identified the most statistically significant biological pathways that were enriched given the set of DEGs in each experiment.

### Differential Survival and Seizure Frequency for Untreated and Treated Mice:

Adults: Table 1 displays survival and seizure statistics for female and male untreated (N=17) and treated (n=10) B6-D/+ monitored 24/7 by video beginning at P30 and continuing for the remainder of their entire life span. Data for the untreated mice were previously reported. Table 1 also provides C3H-D/D summary statistics for female untreated (N=15) and treated (N=15) and male untreated (N=27) and treated (N=16) monitored until the time of death. Age at seizure onset was not significantly different for untreated and treated mice within each line, except in the case of male C3H-D/D (44.0+6.5 days vs 48.1+5.1 days, t-test P-value=0.018).

TABLE 1
Mortality and morbidity summary statistics for untreated versus treated adult
mice followed for the entire lifespan.

| | | | | | #TCs/ | | #Days | | mean | |
|---|---|---|---|---|---|---|---|---|---|---|
| | | | Age at | | day | Age at | survived | #gaps | gap | gap |
| | | N | Death | #TCs | (PostTC) | Onset | postTC | (av) | length | days |
| B6 | Untreated | 17 | 132.9 | 91.9 | 2.11 | 85.6 | 46.7 | 5.1 | 5.0 | 25.7 |
| Females | | | (21.4) | (35.0) | (0.75) | (13.8) | (20.5) | (2.2) | (1.3) | (13.9) |
| (D/+) | Treated | 10 | 167.6 | 14.3 | 0.24 | 91.2 | 76.4 | 4.0 | 8.3 | 34.0 |
| | | | (19.1) | (16.7) | (0.34) | (12.8) | (17.9) | (2.5 | (1.6) | (22.8) |
| | p-value | | **** | **** | **** | ns | *** | ns | *** | **** |
| B6 | Untreated | 17 | 91.3 | 31.7 | 2.3 | 76.2 | 15.1 | 2.4 | 3.1 | 6.2 |
| Males | | | (15.2) | (36.3) | (1.2) | (8.4) | (14.0) | (2.0) | (2.2) | (9.0) |
| (D/+) | Treated | 10 | 135.8 | 43.6 | 1.0 | 81.3 | 52.7 | 4.0 | 12.4 | 37.8 |
| | Late | | (28.4) | (15.8) | (0.5) | (9.1) | (22.3) | (1.2) | (7.6) | (21.3) |
| | p-value | | **** | ns | *** | ns | **** | * | **** | **** |
| | Treated | 13 | 121.6 | 18.9 | 2.4 | 99.2 | 16.8 | 2.6 | 6.7 | 15.0 |
| | Early | | (23.4) | (16.7) | (1.7) | (9.2) | (15.4) | (1.9) | (7.8) | (12.0) |
| | p-value | | **** | ns | ns | **** | ns | ns | * | * |
| C3H | Untreated | 15 | 69. | 26.5 | 2.35 | 47.5 | 22.5 | 3.1 | 4.6 | 15.3 |
| Females | | | (12.9) | (11.6) | (4.0) | (7.0) | (10.8) | (1.1) | (2.9) | (10.1) |
| (D/D) | Treated | 15 | 117.4 | 30.1 | 0.51 | 48.5 | 68.9 | 7.5 | 5.8 | 44.0 |
| | | | (28.0) | (11.2) | (0.29) | (4.4) | (29.2) | (3.0) | (2.3) | (27.6) |
| | p-value | | **** | ns | * | ns | **** | **** | ns | *** |
| C3H | Untreated | 27 | 67.9 | 22.7 | 1.1 | 44.0 | 23.9 | 4.6 | 3.6 | 15.1 |
| Males | | | (9.9) | (8.0) | (0.48) | (6.5) | (11.5) | (2.4) | (1.7) | (9.0) |
| (D/D) | Treated | 16 | 117.1 | 37.4 | 0.57 | 48.1 | 68.9 | 9.8 | 4.7 | 44.4 |
| | | | (20.4) | (13.1) | (0.024) | (5.1) | (19.8) | (4.2) | (1.8) | (22.2) |
| | p-value | | **** | **** | *** | * | **** | **** | * | **** |

t-test or Fisher exact test p-values: ≤0.05 *; ≤0.01 **; ≤0.001 ***; ≤0.0001 ****

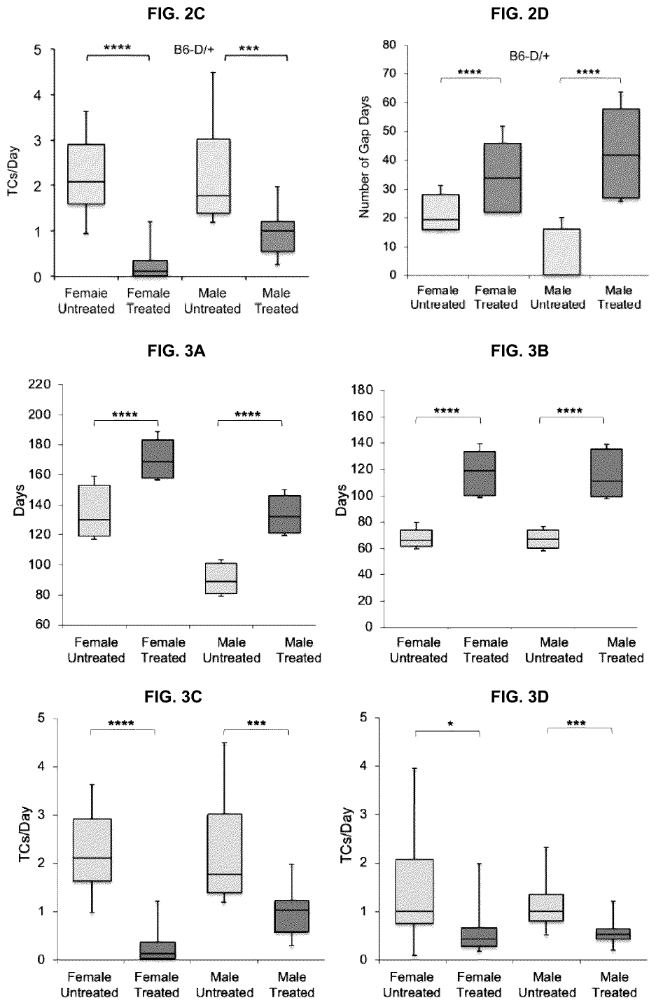

In all cases, treated mice lived longer and experienced a lower post-onset seizure frequency. For example, there was a significant increase in survival of B6-D/+ treated versus untreated females (167.6±19.1 vs 132.9±21.4 days, t-test P-value=1.38×10⁻⁴) and B6-D/+ treated versus untreated males (135.8±28.4 vs 91.3±15.2 days, t-test P-value=<1.0×10⁻⁵), reflecting a 34.7% and 48.9% increase, respectively (FIG. 2A and Table 1). Similarly, C3H-D/D treated versus untreated females (69.9±12.9 vs 117.4±28.0 days, t-test P-value=<1.0×10⁻⁵) and C3H-D/D treated versus untreated males (67.9±9.9 vs 117.1±20.4 days, t-test P-value=<1.0 ×10⁻⁵), reflecting a 47.5% and 49.2% increase, respectively (FIG. 3A-3D and Table 1).

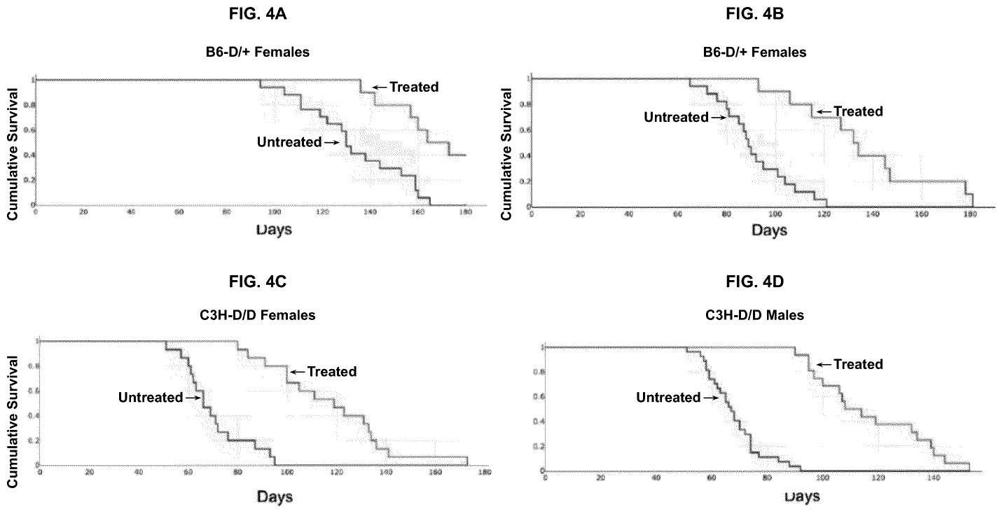

Kaplan-Meier survival curves are shown for all adults in FIG. 4A-4D and for C3H-D/D in FIG. 2B. Unequal survival of treated versus untreated B6-D/+ females and males was strongly supported in a goodness of fit test using the x2 distribution (right-tailed) (P-value=2.1×10⁻³ and 3.5×10⁻⁴, respectively), which also indicated a medium and large observed standard effect sizes of 0.59 and 0.69, respectively (FIG. 4A-4B). C3H-D/D treated females and males showed similar patterns, with unequal survival strongly supported (P-values=<1.0×10⁻⁵ in both cases) with large observed standard effect sizes of 0.83 (FIG. 4C) and 0.79, respectively (FIG. 2B).

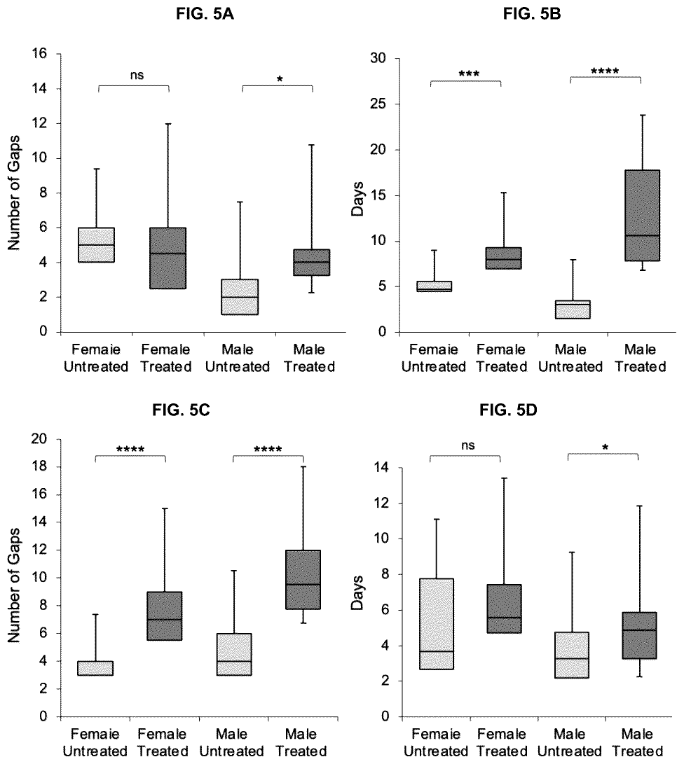

Treated adult mice also had a lower post-onset seizure frequency, with B6-D/+ females and males experiencing 88.9% and 59.1% reductions, respectively (t-test P-value=<1.0 ×10⁻⁵ and 9.4×10⁻⁴, respectively) (FIG. 2C); and C3H-D/D females and males experiencing 78.4% and 48.0% reductions, respectively (t-test P-value=4.2×10⁻² and =1.2×10⁻⁴, respectively) (FIG. 3D and Table 1). These reductions are reflected in a greater number of post-onset seizure gaps (i.e., ≥3 seizure-free days) for B6-D/+ males (t-test P-value=3.9×10⁻²) and C3H-D/D females (t-test P-value=<1.0×10⁻⁵) and males (t-test P-value=<1.0×10⁻⁵) (FIG. 5A-5D and Table 1). In addition, there was a greater gap length in B6-D/+ females (t-test P-value=1.6×10⁻⁴) and males (t-test P-value=3.6×10⁻⁵), as well as in C3H-D/D males (t-test P-value=3.3×10⁻²) (FIG. 3A-3D and Table 1), resulting in a greater number of ‘gap days’ (shown for B6-D/+in FIG. 2D).

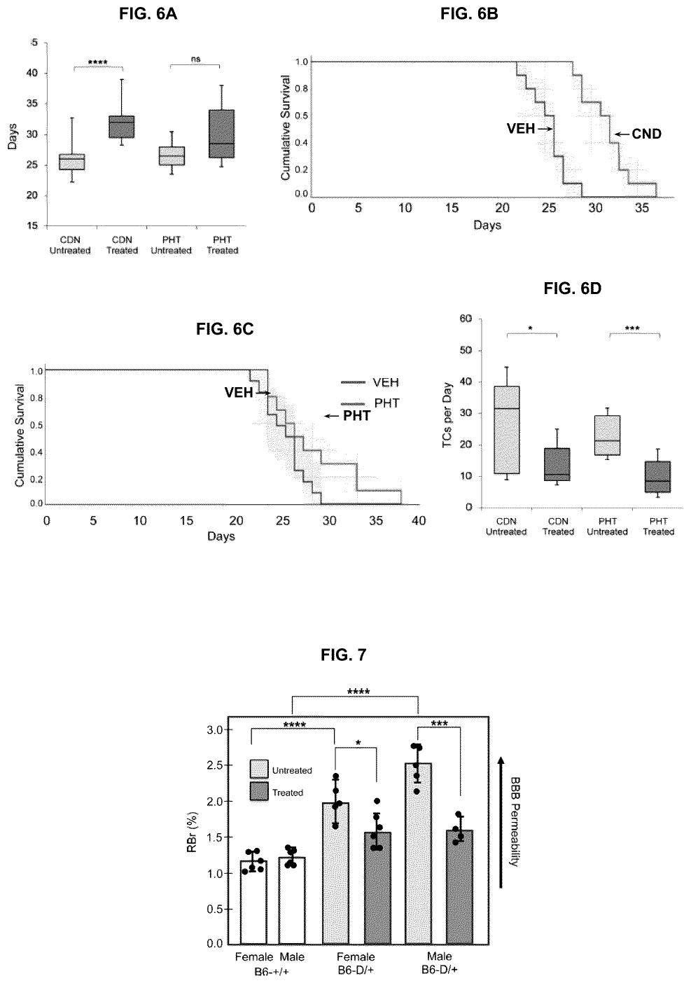

Juveniles: Table 2 displays survival and seizure statistics for two groups of untreated and treated B6-D/D juvenile mice: a set treated with CND (N=10) and VEH (N=10) and a second set treated with PHT (N=10) and VEH (N=10). There was a significant increase (27%) in the survival of juveniles treated with CND (31.8±2.7 vs 25.5±2.1 days, t-test P-value=<1.0×10⁻⁵) (FIG. 6A; Table 2). Juveniles treated with PHT experienced a minor increase in survival that did not reach statistical significance (30.1±5.1 days vs 27.6±3.2) (t-test, P-value=0.083). Kaplan-Meier survival curves similarly show unequal survival of CND treated versus untreated juveniles (P-value =4.2×10⁻⁵) with a large observed standard effect size of 0.79 (FIG. 6B), while PHT treated juveniles did not show a statistically significant increase in survival (P-value=0.132) (FIG. 6C). For juveniles treated with CND there was a slight decrease in post-onset seizure frequency (15.4±10.3 vs 28.4±17.7 TCs, t-test, P-value=2.9×10⁻²), and a more robust reduction in post-onset seizure frequency for juveniles treated with PHT (10.2±7.1 vs 24.8±11.0 TCs, t-test, P-value=7.4 ×10⁻⁴) (FIG. 6D; Table 2).

Table 2 shows the mortality and morbidity summary statistics for B6-D/D juveniles treated with CDN versus PHT.

| | | | | #TCs/ | | #TCs/ | | Days | | mean | |
|---|---|---|---|---|---|---|---|---|---|---|---|
| | | Age at | | day | Age at | day | Age at | with | | gap | gap |
| | | Death | #TCs | (Life) | Onset | (PostTC) | Dosing | TCs | #gaps | length | days |
| CDN | Vehicle | 25.5 | 76.4 | 3.0 | 21.0 | 28.4 | 18.7 | 3.8 | 0.3 | 1.4 | 1.5 |
| | | (2.1) | (29.9) | (1.1) | (3.0) | (17.7) | (2.9) | (1.6) | (0.5) | (2.4) | (2.4) |
| | Treated | 31.8 | 119.0 | 0.09 | 21.4 | 15.4 | 20.0 | 7.2 | 0.9 | 3.4 | 4.3 |
| | | (2.7) | (28.3) | (1.1) | (5.5) | (10.3) | (2.9) | (3.9) | (1.0) | (3.6) | (4.1)( |
| | p-value | **** | ** | * | ns | * | ns | * | * | ns | * |
| PHT | Vehicle | 27.6 | 79.6 | 2.9 | 24.0 | 24.8 | 23.8 | 4.4 | 0.5 | 2.1 | 2.4 |
| | | (3.2) | (24.8) | (0.9) | (3.4) | (11.0) | (3.5) | (1.7) | (0.7) | (2.6) | (3.1) |
| | Treated | 30.1 | 55.9 | 1.9 | 22.2 | 10.2 | 22.2 | 6.8 | 1.2 | 3.5 | 5.2 |
| | | (5.1) | (26.0) | (0.8) | (2.5) | (7.1) | (2.1) | (3.6) | (1.0) | (3.6) | (5.9) |
| | p-value | ns | * | *** | ns | *** | ns | * | * | ns | ns |

t-test or Fisher exact test p-values: ≤0.05 *; ≤0.01 **; ≤0.001 ***; ≤0.0001 ****

Effect of CND on blood-brain barrier permeability in female and male adults: Previously, it was demonstrated that BBB paracellular permeability (i.e., “leak”) to [14C] sucrose, a small molecule tracer that does not cross the intact BBB, increased in both pre-TC B6-D/+ females and males compared to wild-type controls. The magnitude of the sucrose permeability increase was shown to further increase over wild-type and pre-seizure levels in untreated post-TC B6-D/+ females and males. Treatment with CND before seizure onset prevents BBB paracellular permeability from increasing above physiological levels in both B6-D/+ females and males (data not shown). After seizure onset, treatment with CND results in a statistically significantly reduced BBB paracellular permeability (i.e., ‘leak’) relative to untreated post-seizure females (P<0.05) and untreated post-seizure males (P<0.001) (FIG. 7).

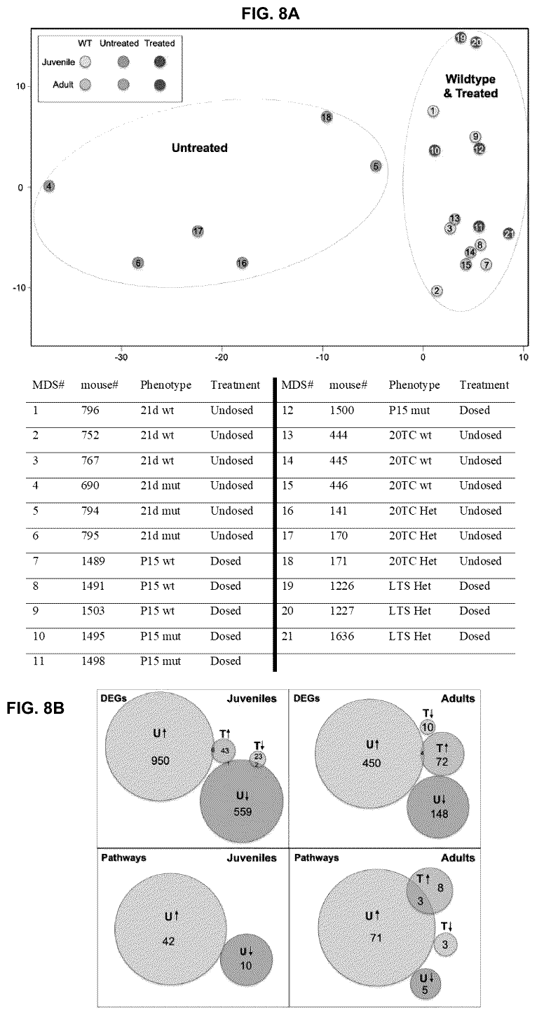

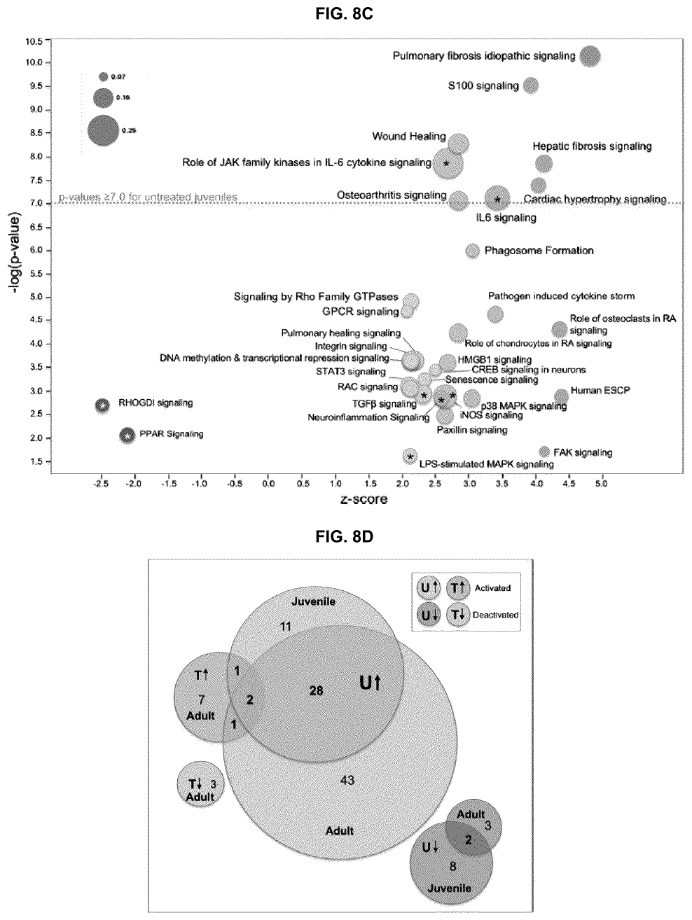

Effects of CND on hippocampal gene expression in juvenile and adult mice: FIG. 8A shows an MDS plot of the 500 most variably expressed genes for the 21 B6-D/D and B6-D/+ individuals submitted to RNAseq analysis. The plot shows untreated juveniles and adults on the left side of the plot and CND-treated juveniles and adults clustering on the right side with age-matched wild-type individuals. The co-clustering of juvenile and adult mice on both sides of the plot suggests that treatment with CND is the chief explanatory factor (i.e., more important than age or genotype) underlying inter-group genome-wide gene expression differences.

To further investigate the effects of CND treatment on gene expression in mice experiencing TCs, the number of DEGs that are up-regulated and down-regulated were characterized in the untreated and treated groups. For untreated juveniles, 1518 transcripts were identified with an FDR-adjusted P-value <0.05, 956 of which were up-regulated and 562 of which were down-regulated (FIG. 8B, upper panel). CND-treated B6-D/D mice had a much smaller number of DEGs: 50 up-regulated and 25 down-regulated. Only 6 of the up-regulated and 2 of the down-regulated transcripts were shared with untreated mice. Untreated B6-D/+ adult males had a total of 602 DEGs (FDR-adjusted P-value <0.05), 454 of which were up-regulated and 148 were down-regulated. CND-treated B6-D/+ males had 86 DEGs: 76 up-regulated and 10 down-regulated. Four upregulated transcripts were shared between untreated and treated males (FIG. 8B, upper panel).

Canonical pathways altered in untreated and treated juvenile mice: For untreated B6-D/D juveniles, pathway enrichment procedures identified 52 canonical pathways with P-values ≤0.05 and z-scores with absolute values≥2.0, including 10 predicted to be deactivated and 42 predicted to be activated (FIG. 8B, lower panel). There were no enriched canonical pathways for CND-treated B6-D/D under strict FDR. After relaxing the FDR cutoff from 0.05 to 0.25 a set of related pathways became statistically significant in treated juveniles: Oxidative Phosphorylation (−log (P-value)=9.9, z-score=5.0), EIF2 Signaling (−log (P-value)=4.2, z-score=3.2), Mitochondrial Dysfunction (−log (P-value)=7.6, z-score=−3.8), and Granzyme A Signaling (−log (P-value)=5.5, z-score=−3.4). The enrichment results reflect a common set of 19 up-regulated electron transport chain (ETC) subunit genes that alternately result in positive and negative activation z-scores for the oxidative phosphorylation (OXPHOS) and mitochondrial dysfunction (MD) pathways, respectively (another 24 non-ETC genes distinguish the MD from OXPHOS pathways). These genes include 12 within ETC complex I (Ndufa2, Ndufa4, Ndufa6, Ndufb4, Ndufb6, Ndufs3, Ndufs4, Ndufs6, Ndufs7, Ndufs8, Ndufv1, and Ndufv2), 3 within ETC complex III (Uqcrc1, Uqcr2 and Uqcr11), and 4 within ETC complex IV (Cox6c, Cox7a2, Cox7b, and Surf1). Only a single transcript out of 96 ETC genes was differentially expressed in untreated juveniles; the up-regulation of UCP2 (+3.32-fold, P-value=1.39×10⁻⁸, z-score=1.06 ×10⁻⁵) has been shown to shift metabolism away from OXPHOS toward glycolysis. A Fisher exact test comparing the number of differentially expressed ETC genes (FDR≤0.25) in treated versus untreated yields a P-value of 1.64×10⁻⁵, and results in an odds ratio representing a 23-fold enhancement of ETC gene activation in treated juveniles (95% CI=3.5-979).

Canonical pathways altered in untreated and treated adult mice: For untreated B6-D/+ adults, pathway enrichment procedures identified 79 canonical pathways with P-values≤0.05 and z-scores with absolute values≥2.0, including 5 predicted to be deactivated and 74 predicted to be activated (FIG. 8B, lower panel). Treatment with CND resulted in an enrichment of 14 canonical pathways: 3 predicted to be deactivated and 11 predicted to be activated. Only three activated pathways were shared between untreated and treated B6-D/+ males. Eight pathways uniquely activated in treated adults are involved in T-helper 1 cell-mediated immune responses, 5 of which involve Regulatory T cells (Tregs) (Dendritic Cell Maturation, Calcium-induced T Lymphocyte Apoptosis, ICOS-ICOSL Signaling in T Helper Cells, PKCO Signaling in T Lymphocytes, T Cell Receptor Signaling).

Altered canonical pathways shared between untreated juvenile and adult mice: FIG. 8C shows a bubble chart of the 30 canonical pathways shared between untreated B6-D/D juveniles and B6-D/+ adults. Of the 30 shared enriched pathways, 2 are predicted to be deactivated (PPAR and RHOGDI Signaling), and 28 are predicted to be activated (FIG. 8D). The top enriched pathways (i.e., −log (P-value)>7.0) that are shared between juvenile and adults include Pulmonary Fibrosis Idiopathic Signaling, Osteoarthritis Pathway, Hepatic Fibrosis Signaling, S100 Family Signaling, Wound Healing Signaling, Role of JAK family kinases in IL-6-type Cytokine Signaling, and IL-6 Signaling.

Of the pathways uniquely altered in untreated juveniles, 8 were predicted to be deactivated and 11 were predicted to be activated (FIG. 8D). Of those uniquely altered in untreated adults, 3 are predicted to be deactivated and 43 predicted to be activated (FIG. 8D). None of the abovementioned pathways was found to be activated in juveniles or adult males treated with CND.

Predicted upstream regulator molecules: To identify potential drivers of the differential expression pattern observed within each dataset, we used the upstream regulator function in IPA. Lipopolysaccharide (LPS), TNF, IL1B, and transforming growth factor-β (TGF-β) were the top predicted upstream activators in both untreated juveniles and adult males (including their rank order), with activation z-score ranging from 7.1 to 9.6 and 6.8 to 8.6, respectively (Table 3). None of the z-scores was statistically significant for these upstream activators in either juvenile or adults treated with CND (Table 3). Similarly, neither of the top predicted upstream inhibitors (also shared between) untreated juvenile and adults-APOE (z-scores: −4.0 and −4.6, respectively) and PPARGCIA (z-scores: −2.4 and −3.8, respectively)-reached statistical significance in treated juveniles and adults (Table 3). While not appearing on the list of upstream regulators in juveniles, the top predicted upstream inhibitor for treated adults (PTGS2 or cyclooxygenase-2, z-score=-2.8) was oppositely predicted to be an upstream activator in the case of untreated adults (z-score=2.5).

Table 3 shows top predicted upstream regulators

| | UNTREATED | | Untreated | TREATED | | |
|---|---|---|---|---|---|---|
| | p-value | z-score | State | p-value | z-score | Treated State |
| B6-D/D Juveniles | | | | | | |
| Top Activators | | | | | | |
| Untreated | | | | | | |
| lipopolysaccharide | 4.63E−51 | 9.59 | Activated | 4.41E−06 | 3.19 | Activated |
| TNF | 4.67E−47 | 7.07 | Activated | 4.36E−03 | 1.32 | normalized |
| IL1B | 3.16E−38 | 7.43 | Activated | 3.33E−03 | 1.76 | normalized |
| TGFB1 | 3.38E−37 | 9.39 | Activated | 7.70E−05 | 0.42 | normalized |
| Top Inhbitors | | | | | | |
| Untreated | | | | | | |
| APOE | 4.87E−11 | −3.96 | Deactivated | na | na | normalized |
| PPARGC1A | 3.54E−14 | −2.44 | Deactivated | na | na | normalized |
| Top Inhibitor | | | | | | |
| Treated | | | | | | |
| PTGER4 | 3.01E−06 | ns | null | 1.04E−06 | −2.43 | Deactivated |
| ACKR2 | na | na | null | 3.64E−06 | −2.00 | Deactivated |
| SIRT1 | 1.32E−07 | na | null | 5.21E−06 | −2.83 | Deactivated |
| B6-D/+ Adult males | | | | | | |
| Top Activators | | | | | | |
| Untreated | | | | | | |
| lipopolysaccharide | 4.41E−50 | 8.56 | Activated | 1.55E−06 | ns | normalized |
| TNF | 4.33E−45 | 6.79 | Activated | 9.93E−04 | ns | normalized |
| IL1B | 2.70E−43 | 7.08 | Activated | 3.76E−04 | ns | normalized |
| TGFB1 | 3.98E−39 | 7.19 | Activated | 8.07E−08 | ns | normalized |
| Top Inhbitors | | | | | | |
| Untreated | | | | | | |
| APOE | 4.87E−15 | −4.62 | Deactivated | 3.35E−02 | ns | normalized |
| PPARGC1A | 2.44E−14 | −3.78 | Deactivated | ns | ns | normalized |
| Top Inhibitor | | | | | | |
| Treated | | | | | | |
| PTGS2 | 8.02E−12 | 2.51 | Activated | 6.99E−08 | −2.80 | Deactivated |
| ERBB2 | 6.53E−20 | 3.49 | Activated | 2.15E−04 | −2.00 | Deactivated |
| NUPR1 | 3.07E−05 | 2.32 | Activated | 8.66E−02 | −2.00 | Deactivated |

pval: p-value;
zsc: z-score;
ns: not significant;
N/A: not called

The present invention features the first study to test CND administration in a mouse model with a natural onset of seizures and to investigate the cellular and molecular mechanisms involved in improved outcomes. The results indicate significant efficacy of CND from three perspectives: survival and seizure frequency, BBB function, and genome-wide hippocampal gene expression. The findings of extended lifespan, longer seizure-free periods, and reduced seizure frequency in Scn8a-N1768D mice treated with CND are robust to age, sex, and strain background (FIG. 2A-2D and FIG. 6A-6D). Treatment with CND yielded greater increases in survival, with B6-D/+ males surviving for a mean of 44.5 days longer (48.9%, t-test p-value=<1.0×10⁻⁵) and B6-D/D males surviving for 6.3 days longer (27%, t-test, P-value=<1.0×10⁻⁵). These increases represent larger standard effect sizes of 0.69 and 0.79, respectively, compared with 0.50 and 0.35 for CBD-treated B6-D/+ and B6-D/D males. In addition, CND treatment resulted in greater reductions in seizure frequency for B6-D/+ males (56.5%) and B6-D/D juveniles (45.8%). The present study found CND also outperformed phenytoin—an antiseizure medication shown to be efficacious for patients with SCN8A, in terms of survival in juvenile Scn8a-D/D mice.

Adult B6-D/+ females and males both exhibit significantly reduced BBB ‘leak’ when treated with CND after the establishment of seizures (FIG. 7). Untreated B6-D/D juvenile and B6-D/+ adult males exhibited significant ‘disease-induced’ genome-wide changes in hippocampal transcript abundance, many of which were returned to physiological levels in treated mice. Characterization of enriched pathways in untreated mice revealed a host of cellular and molecular processes representing both adaptive and maladaptive responses to increased neuronal excitability.

Epilepsy is a common neurological disorder, yet few, if any, currently marketed antiseizure medications are capable of preventing or curing the condition. As shown herein, angiotensin receptor blockers (ARBs) offer a promising therapeutic approach to mitigate the numerous detrimental effects of epileptogenesis. Notably, Scn8a pathology mirrors the effects of angiotensin type 1 receptor (AT1R) overstimulation, suggesting that blocking AT1R activation could provide therapeutic benefits in this model and other neurological disorders with similar pathological features. Testing candesartan, an FDA-approved ARB indicated for hypertension in pediatric patients over one year old, in a transgenic Scn8a mouse model has demonstrated strong efficacy.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of treating an epileptic condition in a patient in need thereof, the method comprising administering a therapeutically effective amount of an angiotensin receptor blocker (ARB) to the patient.

2. The method of claim 1, wherein the epileptic condition is pediatric epilepsy, a traumatic brain injury (TBI), Alzheimer's disease (AD), Parkinson's disease (PD), amyotrophic lateral sclerosis (ALS), Huntington's disease (HD), stroke, post-traumatic epilepsy, and temporal lobe epilepsy (TLE).

3. The method of claim 1, wherein the ARB is a sartan.

4. The method of claim 3, wherein the sartan is candesartan, losartan, valsartan, irbesartan, telmisartan, eprosartan, azilsartan, olmesartan, or derivatives thereof.

5. The method of claim 1, wherein the method reduces seizure frequency.

6. The method of claim 1, wherein the ARB is administered orally.

7. The method of claim 1, wherein the ARB is administered daily.

8. A method of treating epilepsy in a patient in need thereof, the method comprising administering a therapeutically effective amount of an angiotensin receptor blocker (ARB) to the patient.

9. The method of claim 8, wherein the ARB is a sartan.

10. The method of claim 9, wherein the sartan is candesartan, losartan, valsartan, irbesartan, telmisartan, eprosartan, azilsartan, olmesartan, or derivatives thereof.

11. The method of claim 8 wherein the patient is a child.

12. The method of claim 11, wherein the epileptic condition is pediatric epilepsy.

13. The method of claim 8, wherein the method reduces seizure frequency.

14. The method of claim 8, wherein the ARB is administered orally.

15. The method of claim 8, wherein the ARB is administered daily.

16. A method of protecting the blood-brain barrier (BBB) in a subject in need thereof, the method comprising administering a therapeutically effective amount of an angiotensin receptor blocker (ARB) to the subject.

17. The method of claim 16, wherein the ARB is a sartan.

18. The method of claim 17, wherein the sartan is candesartan, losartan, valsartan, irbesartan, telmisartan, eprosartan, azilsartan, olmesartan, or derivatives thereof.

19. The method of claim 16, wherein the ARB is administered orally.

20. The method of claim 16, wherein the ARB is administered daily.
